(19) United States
(12) Patent Application Publication
(10) Pub. No.: US 2016/0128647 A1
(43) Pub. Date: May 12, 2016

(54) Medical Device With Enhanced Viewing Mode

(71) Applicant: Welch Allyn, Inc., Skaneateles Falls, NY (US)

(72) Inventors: Catherine M. King, Ithaca, NY (US); Thomas A. Myers, Syracuse, NY (US); Shawn C. St. Pierre, Syracuse, NY (US)

(21) Appl. No.: 14/535,547

(22) Filed: Nov. 7, 2014

(51) Int. Cl. A61B 5/00

(52) U.S. Cl. CPC A61B 5/7475; A61B 5/742

(57) ABSTRACT

A medical device includes: a processor; a display; and memory encoding instructions that, when executed by the processor, cause the processor to create an interface on the display, the interface including: a plurality of vital signs data positioned on the interface, with each vital sign being displayed in an enlarged font and being listed in order of importance; and a control for accessing other functionality of the medical device.

### BACKGROUND

As the amount of information that is captured by medical devices increases, the complexity of using the devices also increases. This complexity extends into how the data is displayed to the user. In some interfaces, the amount of data that is provided can function to obscure important information, particularly when the display is viewed at a distance.

### SUMMARY

In one aspect, a medical device includes: a processor; a display; and memory encoding instructions that, when executed by the processor, cause the processor to create an interface on the display, the interface including: a plurality of vital signs data positioned on the interface, with each vital sign being displayed in an enlarged font and being listed in order of importance; and a control for accessing other functionality of the medical device.

In another aspect, a medical device includes: a processor; a display; and memory encoding instructions that, when executed by the processor, cause the processor to create a lock screen interface on the display, the lock screen interface including: a plurality of vital signs data positioned on the lock screen interface, with each vital sign being displayed in an enlarged font and being listed in a vertical orientation in order of importance; and a control for unlocking the medical device.

In yet another aspect, a medical device includes: a processor; a display; and memory encoding instructions that, when executed by the processor, cause the processor to create a lock screen interface on the display, the lock screen interface including: a plurality of vital signs data positioned on the lock screen interface, with each vital sign being displayed in an enlarged font and being listed in a vertical orientation in order of importance, wherein a font size for one or more of the vital signs data is varied, and wherein a font color for one or more of the vital signs data is varied; and a control for unlocking the medical device.

### DESCRIPTION OF THE FIGURES

FIG. 1 illustrates a block diagram of a wireless ambulatory care system.

FIG. 2 illustrates an example medical device of the system of FIG. 1.

FIG. 3 illustrates another example medical device of the system of FIG. 1.

FIG. 4 illustrates an example user interface for the medical device of FIG. 3.

FIG. 5 illustrated another example user interface for the medical device of FIG. 3.

FIG. 6 is a block diagram illustrating physical components of a computing device with which examples and embodiments of the disclosure can be practiced.

### DETAILED DESCRIPTION

Examples of ambulatory care environments can include hospitals, clinics, managed care facilities, and other locations where medical care is provided. Medical personnel in ambulatory care environments can utilize vital signs monitoring devices, vital signs displays, personal computing devices and electronic medical record access portals. Medical staff and providers often need to record a patient's vital signs and enter those vital signs into the patient's electronic medical record. Currently, providers must perform vital signs measurements, remember the measurements, and then enter those measurements into one or more computing devices which may or may not be directly linked to the patient's electronic medical record.

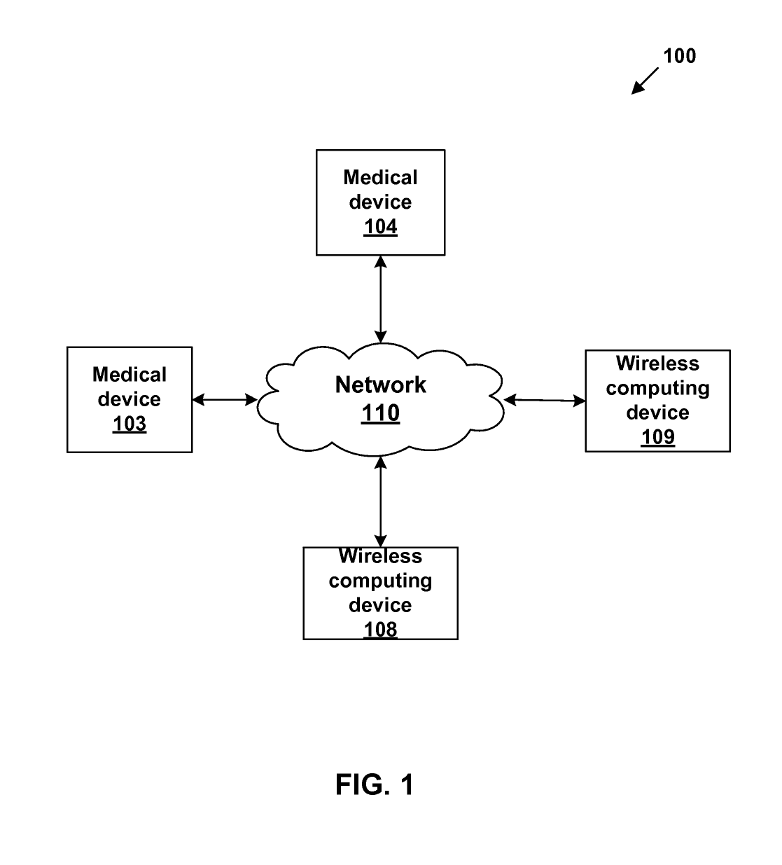

FIG. 1 illustrates a block diagram of an example wireless ambulatory care network 100. The example network 100 includes medical devices 103 and 104, wireless computing devices 108 and 109, and communication network 110. In embodiments, the example network 100 can include more or fewer medical devices 103 and 104. In embodiments, the example network can include more or fewer wireless computing devices 108 and 109. The communication network 110 can be a wireless network, such as WiFi, Bluetooth, Zigbee, Ant, Z-Wave, etc.

In some embodiments, the one or more medical devices 103 and 104 can include one or more vital signs measurement components. For example, the medical devices 103 can include, for example, a thermometer, a heart rate monitor, a pulse oximeter, a non-invasive blood pressure monitor, and a respiration rate monitor. In embodiments, one or more vital signs measurement components are wirelessly linked to the medical devices 103 and 104 and can transmit measurements to the medical devices 103 and 104.

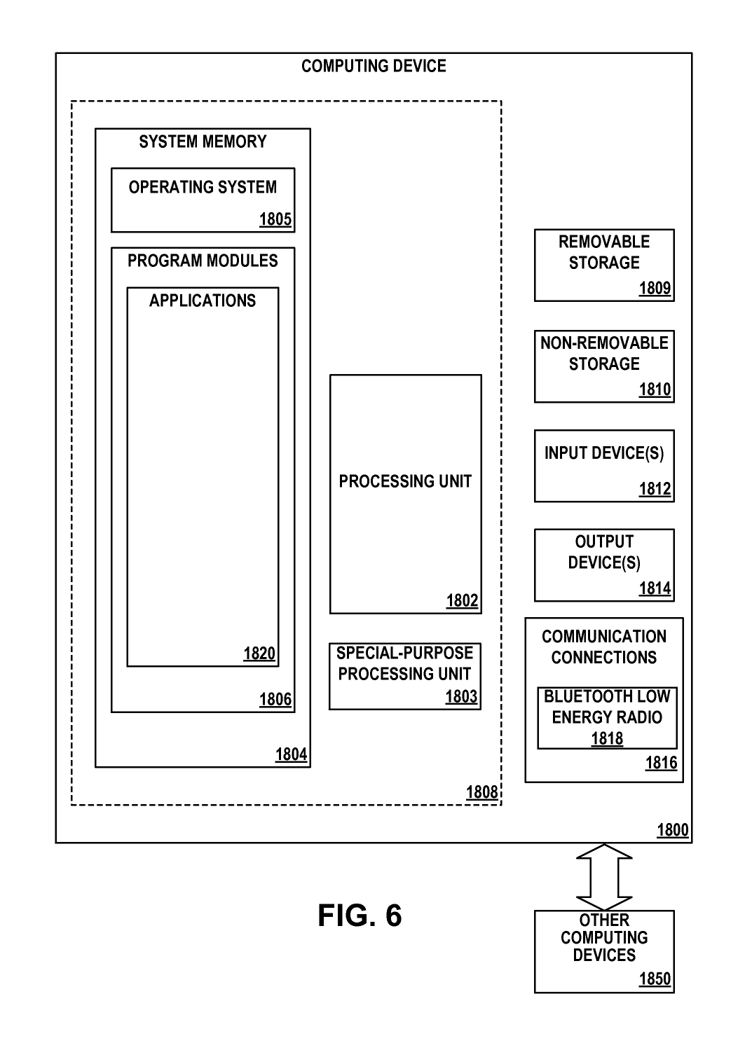

Example computing components of medical devices 103 and 104 are shown and described in more detail with reference to FIG. 6, below.

In some embodiments, the one or more wireless computing devices 108 and 109 can be smart phones, tablet computers, personal digital assistants, laptop computers, and desktop computers, which can optionally be mounted on portable carts. Example computing components of the one or more wireless computing devices 108 and 109 are shown and described in more detail with reference to FIG. 6, below. The use of less complicated wireless computing devices 108 and 109, such as heart rate monitors, pulse oximeters, etc., is also contemplated by this document.

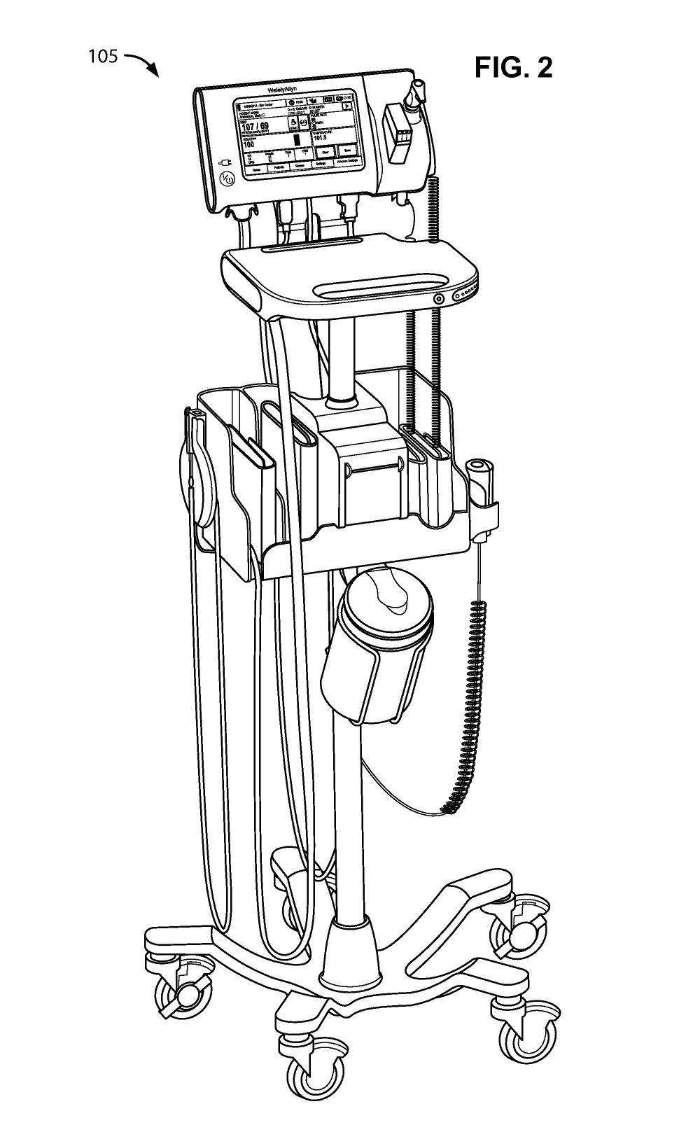

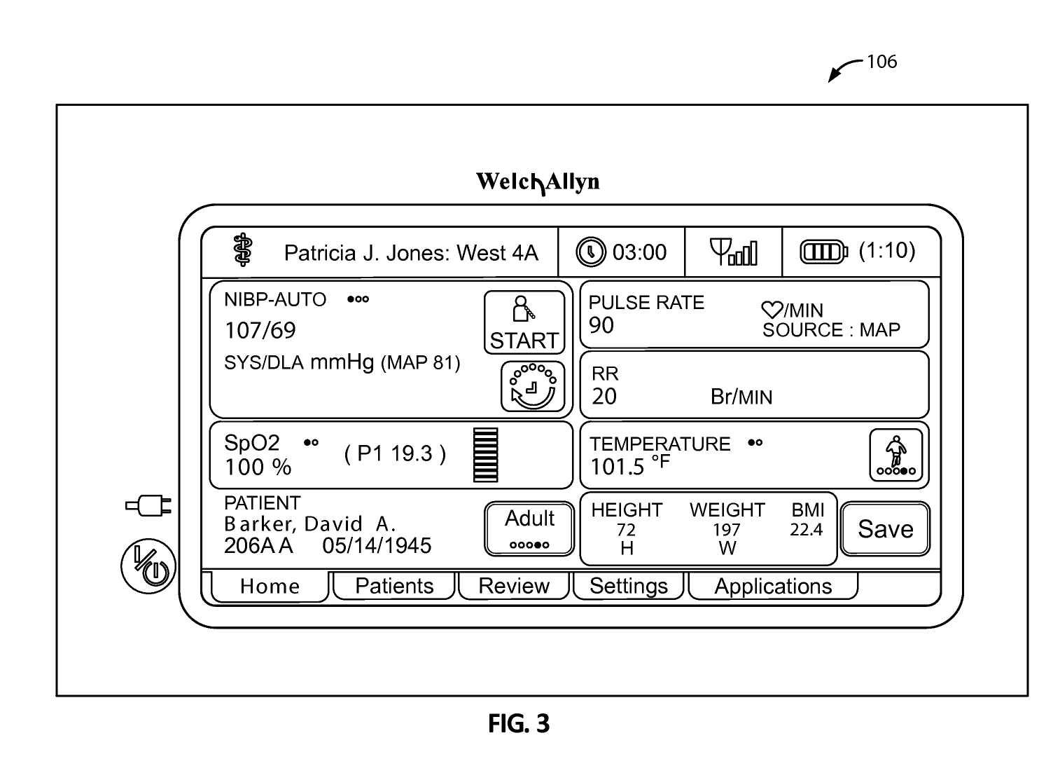

FIG. 2 illustrates one example of the medical device 105. The medical device 105 is shown on a mobile cart, and the medical device 105 is programmed to provide the functionalities described herein. The medical device 105 includes a user interface, such as a touch screen, and includes the ability to execute multiple workflows or profiles. In some embodiments, the medical devices 105 and 106 in FIGS. 2 and 3 are the medical device 103 or 104 shown in, and described with reference to, FIG. 1. Other embodiments can include more or fewer components than those shown in FIG. 2, or include different components that accomplish the same or a similar function.

The medical device 105 is able to operate within one or more profiles. A profile is a series of one or more tasks that a user of the medical device 105 performs. When the medical device 105 operates within a profile, the medical device 105 provides functionality suitable for assisting the user in performing the profile. When the medical device 105 operates within different profiles, the medical device 105 provides different functionality.

When the medical device 105 is manufactured, the medical device 105 is configured to be able to operate within one or more profiles. After the medical device 105 is manufactured, the medical device 105 can be reconfigured to operate within one or more additional profiles. In this way, a user can adapt the medical device 105 for use in different profiles as needed.

In various embodiments, the medical device 105 operates within various profiles. For example, in some embodiments, the medical device 105 can operate within a monitoring profile or a non-monitoring profile. Example types of non-monitoring profiles include, but are not limited to, a spot check profile and an office profile. An example of a monitoring profile includes, but is not limited to, an intervals profile.

An additional example of the medical device 106 is shown in FIG. 3. In this example, the medical device 106 is similar to that of the medical device 105 described above. In embodiments, the medical device 106 is mounted on a wall. The medical device 106 is programmed in a manner similar to that described above to monitor physiological parameters of a patient. In some embodiments, the medical device 106 is a stand-alone device, which can mean that is not part of a mobile cart and it is not part of a wall-mounted station.

In the examples described herein, the medical devices 104, 105, 106 are computing devices that have been programmed to perform special, complex functions. These specially-programmed devices function to manipulate and provide data to the users in an improved form factor and with greater efficiency.

For example, as described further below, the medical devices 104, 105, 106 are specially programmed to provide the user with an improved interface that allows the user to discern important information at a glance. This improved interface removes unnecessary information and controls so that the data that is important can be more efficiently and easily viewed, particularly when the user is positioned at a distance from the medical device.

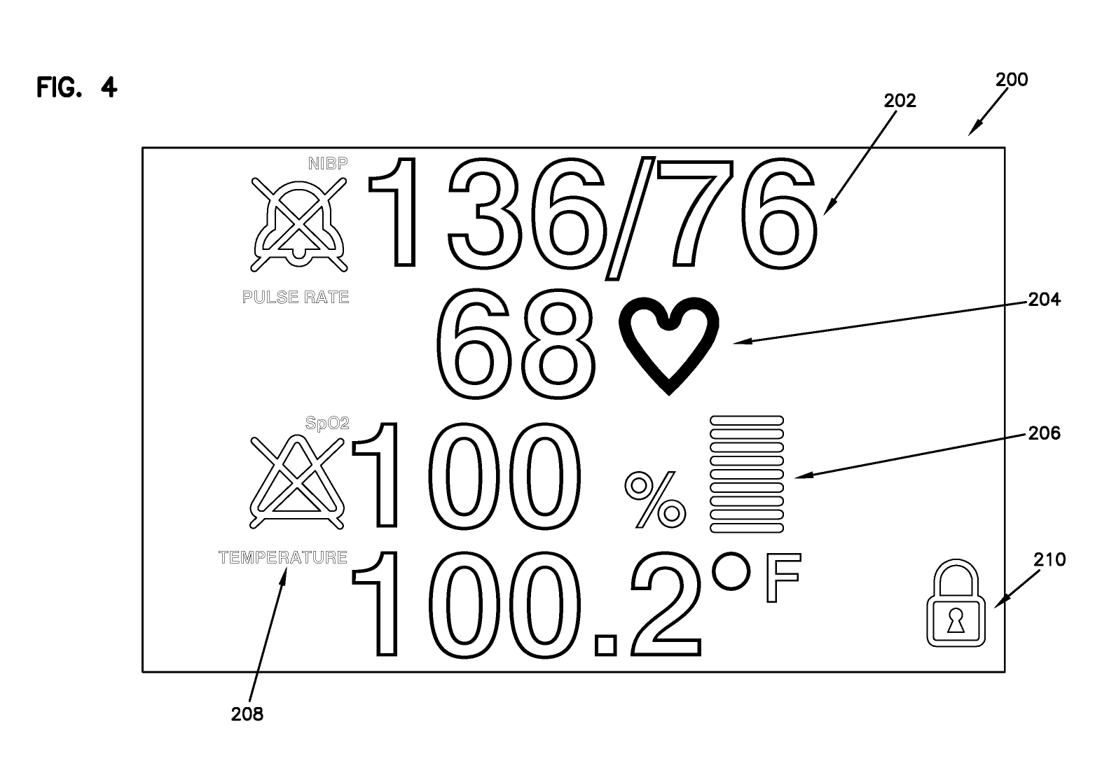

Referring now to FIG. 4, an example user interface 200 is shown. Generally, the interface 200 is enhanced by removing unneeded information and controls and increasing the size of the data that is displayed on the interface 200. In this example, patient and control-related text is removed so that only numeric associated with the vital signs data is shown, along with a short tag for each data type (e.g., NIBP, Pulse Rate, etc.).

Specifically, the interface 200 displays certain vital signs data, including NIBP data 202, pulse rate data 204, SpO2 data 206, and temperature data 208. The vital signs data that is displayed can be chosen based upon importance. For example, a determination can be made that the NIPB data 202 is more important than the temperature data 208 and therefore the NIPB data 202 is placed at the top of the interface 200. In addition, the data displayed itself can be selected so that only important vital signs data is displayed on the interface 200.

As illustrated, the vital signs data is shown in a single vertical column with an increased font (compare to the font size shown in the interface shown in FIG. 3). In such an example, the readability of the data is enhanced. This allows the user to more easily determine which data is most important and also read that data at greater distances from the medical device.

Other enhancements, such as color-coding, can be applied to further optimize readability. For example, each of the vital signs data 202, 204, 206, 208 can be shown in a particular color according to a known color scheme so that a user can easily identify which data is associated with which vital sign. For example, the NIBP data 202 can be depicted in an orange font and the pulse rate data 204 in a green font to allow for even easier recognition by the user.

In some examples, the facility and/or user can control the types of data, size, color, and placement on the interface 200. For example, the user can decide which vital signs data is placed on the interface 200 and how large the font is used for the display. Other configurations are possible.

The interface 200 also reduces or eliminates patient-identifying information on the interface 200, which thereby enhances privacy. As shown, the interface 200 does not provide any patient-identifiers. In other examples, a short-form patient identifier (e.g., name, initials, or number) could be provided.

The interface 200 also reduces controls on the interface 200 so that readability is enhanced. In this example, a single control 210 is shown. This control 210 allows the user to change access other interfaces on the medical device. For example, the control 210 can be used to access the interface shown in FIG. 3, as described below.

In some embodiments, the interface 200 is used as a lock screen for the medical devices 104, 105, 106. In this example, the medical device can be placed in the lock screen when input to the medical device is complete and the medical device is being used to monitor the patient. In such a scenario, the interface of FIG. 3 is replaced with the interface 200 when the medical device is locked. The locking can occur through input by the user (e.g., manually locking the device) and/or through an automated process (e.g., the medical device automatically locks after a certain period of inactivity).

To unlock the device and access an interface that provides further data and controls, the user can select the control 210. Depending on the desired behavior, the user may be prompted for credentials before the device allows the user to clear the interface 200.

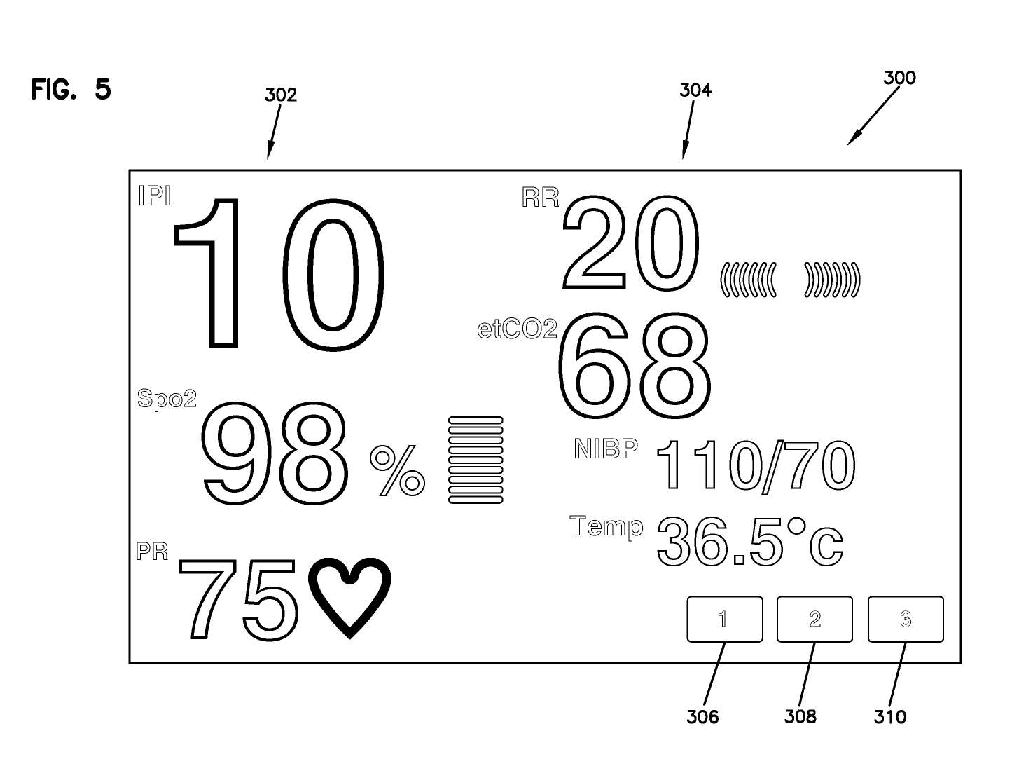

Referring now to FIG. 5, another example interface 300 is shown. In this example, the interface includes columns 302 and 304 of vital signs data. In addition, the font used for the different vital signs data varies depending on placement and importance of the data. (e.g., the size of the font used to display data such as temperature data, integrated pulmonary index (IPI) data, and end-tidal carbon dioxide (ETCO2) data is varied).

In addition, there is a plurality of controls 306, 308, 310 provided on the interface. These controls 306, 308, 310 can be used to manipulate the function of the medical device. For example, one control can be used to unlock the device and change the display back to that shown in FIG. 3. The other controls can be used to access other functionality of the medical device, such as alarming or configuration of the medical device. Finally, these controls can be configured as desired to provide added functionality while still optimizing the display of the most relevant vital signs data for the user.

FIG. 6 is a block diagram illustrating physical components (i.e., hardware) of a computing device 1800 with which embodiments of the disclosure may be practiced. The computing device components described below may be suitable to act as the computing devices described above, such as wireless computing device and/or medical device of FIG. 1. In a basic configuration, the computing device 1800 may include at least one processing unit 1802 and a system memory 1804. Depending on the configuration and type of computing device, the system memory 1804 may comprise, but is not limited to, volatile storage (e.g., random access memory), non-volatile storage (e.g., read-only memory), flash memory, or any combination of such memories. The system memory 1804 may include an operating system 1805 and one or more program modules 1806 suitable for running software applications 1820. The operating system 1805, for example, may be suitable for controlling the operation of the computing device 1800. Furthermore, embodiments of the disclosure may be practiced in conjunction with a graphics library, other operating systems, or any other application program and is not limited to any particular application or system. This basic configuration is illustrated in FIG. 6 by those components within a dashed line 1808. The computing device 1800 may have additional features or functionality. For example, the computing device 1800 may also include additional data storage devices (removable and/or non-removable) such as, for example, magnetic disks, optical disks, or tape. Such additional storage is illustrated in FIG. 6 by a removable storage device 1809 and a non-removable storage device 1810.

Other program modules that may be used in accordance with embodiments of the present disclosure, and in particular to generate screen content, may include electronic mail and contacts applications, word processing applications, spreadsheet applications, database applications, slide presentation applications, drawing or computer-aided application programs, etc.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. For example, embodiments of the disclosure may be practiced via a system-on-a-chip (SOC) where each or many of the components illustrated in FIG. 6 may be integrated onto a single integrated circuit. Such an SOC device may include one or more processing units, graphics units, communications units, system virtualization units and various application functionality all of which are integrated (or “burned”) onto the chip substrate as a single integrated circuit. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

The computing device 1800 may also have one or more input device(s) 1812 such as a keyboard, a mouse, a pen, a sound or voice input device, a touch or swipe input device, etc. The output device(s) 1814 such as a display, speakers, a printer, etc. may also be included. The aforementioned devices are examples and others may be used. The computing device 1800 may include one or more communication connections 1816 allowing communications with other computing devices 1818. Examples of suitable communication connections 1816 include, but are not limited to, RF transmitter, receiver, and/or transceiver circuitry; universal serial bus (USB), parallel, and/or serial ports. Additionally, the communication connections 1816 can include a Bluetooth Low Energy Radio.

The term computer readable media as used herein may include non-transitory computer storage media. Computer storage media may include volatile and nonvolatile, removable and non-removable media implemented in any method or technology for storage of information, such as computer readable instructions, data structures, or program modules. The system memory 1804, the removable storage device 1809, and the non-removable storage device 1810 are all computer storage media examples (i.e., memory storage.) Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 1800. Any such computer storage media may be part of the computing device 1800. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

Embodiments of the present disclosure may be utilized in various distributed computing environments where tasks are performed by remote processing devices that are linked through a communications network in a distributed computing environment.

The logical flows depicted herein are just examples. There may be many variations to these diagrams or the steps (or operations) described therein without departing from the spirit of the disclosure. For instance, the steps may be performed in a differing order, or steps may be added, deleted or modified.

While embodiments have been described, it will be understood that those skilled in the art, both now and in the future, may make various improvements and enhancements can be made.

What is claimed is:

1. A medical device comprising:
a processor;
a display; and
memory encoding instructions that, when executed by the processor, cause the processor to create an interface on the display, the interface including:
a plurality of vital signs data positioned on the interface, with each vital sign being displayed in an enlarged font and being listed in order of importance; and
a control for accessing other functionality of the medical device.

2. The medical device of claim 1, wherein the vital signs data is positioned vertically on the interface.

3. The medical device of claim 2, wherein the vital signs data is listed from most important to least important.

4. The medical device of claim 1, wherein a font size for one or more of the vital signs data is varied.

5. The medical device of claim 1, wherein a font color for one or more of the vital signs data is varied.

6. The medical device of claim 1, wherein only numerical data is displayed on the medical device.

7. The medical device of claim 1, wherein the interface is displayed as a lock screen.

8. The medical device of claim 7, wherein the lock screen is displayed automatically when the medical device is inactive for a period of time.

9. A medical device comprising:
a processor;
a display; and
memory encoding instructions that, when executed by the processor, cause the processor to create a lock screen interface on the display, the lock screen interface including:
a plurality of vital signs data positioned on the lock screen interface, with each vital sign being displayed in an enlarged font and being listed in a vertical orientation in order of importance; and
a control for unlocking the medical device.

10. The medical device of claim 9, wherein a font size for one or more of the vital signs data is varied.

11. The medical device of claim 10, wherein a font color for one or more of the vital signs data is varied.

12. The medical device of claim 11, wherein only numerical data is displayed on the medical device.

13. The medical device of claim 9, wherein a font color for one or more of the vital signs data is varied.

14. The medical device of claim 9, wherein only numerical data is displayed on the medical device.

15. The medical device of claim 9, wherein the lock screen interface is displayed automatically when the medical device is inactive for a period of time.

16. The medical device of claim 9, wherein the vital signs data is listed in two columns.

17. A medical device comprising:
a processor;
a display; and
memory encoding instructions that, when executed by the processor, cause the processor to create a lock screen interface on the display, the lock screen interface including:
a plurality of vital signs data positioned on the lock screen interface, with each vital sign being displayed in an enlarged font and being listed in a vertical orientation in order of importance, wherein a font size for one or more of the vital signs data is varied, and wherein a font color for one or more of the vital signs data is varied; and
a control for unlocking the medical device.

18. The medical device of claim 17, wherein the lock screen interface is displayed automatically when the medical device is inactive for a period of time.

19. The medical device of claim 17, wherein the vital signs data is listed in two columns.

20. The medical device of claim 17, wherein only numerical data is displayed on the medical device.
